(19) United States
(12) Patent Application Publication
(10) Pub. No.: US 2015/0190219 A1
(43) Pub. Date: Jul. 9, 2015

(54) DIAPHRAGM ASSIST DEVICE

(71) Applicants: Curt KOTHERA, Crofton, MD (US); Gregory J. HIEMENZ, Silver Spring, MD (US); William S. KRIMSKY, Bel Air, MD (US); Amit N. SHAH, Bethesda, MD (US)

(72) Inventors: Curt Kothera, Crofton, MD (US); Gregory J. Hiemenz, Silver Spring, MD (US); William S. Krimsky, Forest Hill, MD (US); Amit N. Shah, Bethesda, MD (US)

(21) Appl. No.: 14/413,815

(22) PCT Filed: Jul. 9, 2013
(86) PCT No.: PCT/US13/49726

§ 371 Date: Jan. 9, 2015

Related U.S. Application Data
(60) Provisional application No. 61/669,321, filed on Jul. 9, 2012.
(60) Provisional application No. 61/677,425, filed on Jul. 30, 2012.
(60) Provisional application No. 61/793,362, filed on Mar. 15, 2013.

(51) Int. Cl. A61F 2/02

(52) U.S. Cl. CPC A61F 2/02; A61F 2002/482

(57) ABSTRACT

A diaphragm assist device (DAD) for assistance with diaphragm contraction to facilitate breathing. The device assists with respiration of patients having diaphragmatic dysfunction including those awaiting lung transplants, and those in need of weaning from mechanical ventilation, as well as those who can benefit from an implantable ventilator.

### BACKGROUND OF THE INVENTION

1. Field of the Invention

The present invention generally relates to medical devices for diaphragmatic dysfunction and, more particularly, to a diaphragm assist device (DAD) for assistance with diaphragm contraction and/or expansion in order to facilitate a patient's breathing.

2. Description of Prior Art

During inhalation the diaphragm contracts and enlarges the thoracic cavity. A secondary effect is that the contracting, and lowering diaphragm pushes on the liver, stomach, and other viscera to facilitate expansion of the rib cage. Enlarging the thoracic cavity creates suction that draws air into the lungs. When the diaphragm relaxes, air is exhaled by elastic recoil of the lungs, diaphragm, rib intercostals, and viscera. There are many types of breathing patterns, the parameters of such breathing patterns including respiratory frequency and volume, duration of inhalation, exhalation, pauses, and peak inhalation and exhalation. Other functions of the respiratory system include coughing, sneezing, hiccups, and the ability to hold one's breath.

Diaphragmatic dysfunction, disadvantage and/or inadequacy affects respiration in a large and diverse segment of the population, including people suffering from chronic obstructive pulmonary disease (COPD), interstitial lung disease, certain neuro-muscular disorders (e.g., amyotrophic lateral sclerosis (ALS) and Duchenne muscular dystrophy (DMD)), and those with ventilator-induced diaphragmatic dysfunction (VIDD). Of this group, COPD alone affects an estimated 12 million people in the US and 64 million worldwide. This particular irreversible disease is the leading cause of death due to respiratory disease in the United States and the third most common cause of death overall. A common physiological irregularity resulting from COPD, or emphysema, is hyperinflation of the lungs. This reduces curvature of and lowers the position of the diaphragm, which decreases respiratory function by decreasing the volume change potential. Neuro-muscular disorders and ventilator-induced diaphragmatic dysfunction (VIDD), on the other hand, cause decreased respiratory capacity by diaphragmatic denervation and atrophy. In all of these cases, reliable treatment for the diaphragm, the primary muscle responsible for respiration, does not currently exist.

In COPD, a critical consequence of these disadvantaged diaphragms is the rapid increase in the oxygen cost of breathing. That is, the disadvantaged diaphragm becomes increasingly inefficient and consumes larger proportions of the inspired oxygen from the blood, making less oxygen available to other necessary organs and bodily functions, which subsequently cause numerous other related medical problems and poor quality of life. There has been very little effort to address this vital need. Conventional solutions include oxygen treatment, mechanical ventilators, external compression, and electrical diaphragmatic pacemakers.

In certain neuro-muscular diseases, such as ALS and DMD, and primary lung diseases, the patient's ability to clear secretions and mucous from the airway may decrease significantly. This inability to produce an effective cough may lead to lung collapse (atelectasis), inadequate gas exchange, and infections in the lower respiratory tract.

Mechanical ventilators (that force air into a patient's lungs) are often used to aid breathing, but these devices adversely affect the diaphragm and lead to a host of other medical problems, including ventilator-associated pneumonia (VAP) and pneumothorax.

In addition, external devices have been used for application of abdominal pressure to generate artificial respiration. For example, an external belt-type pneumatic device called the pneumatic abdomino-diaphragmatic belt was conceived by Bragg in England in the late 1930s. McSweeney, C. J., The Bragg-Paul Pulsator in treatment of respiratory paralysis. Br Med J; 1:1206-1207 (1938).

Electrical diaphragmatic pacemakers are typically surgically implanted devices that work by placement of electrodes on the phrenic nerve or diaphragm for electrical pacing.

U.S. Pat. No. 8,281,792 to Royalty issued Oct. 9, 2012 shows an electromagnetic diaphragm assist device that uses a magnetic mat implanted adjacent the diaphragm. The mat compresses the diaphragm in response to application of an electromagnetic field. The device also includes an electromagnetic assembly adapted for surrounding the torso of the human body to toggle the electromagnetic field. Piezoelectric sensors on the mat are used to indicate how much force is being applied to the diaphragm in each direction.

This prior art would be difficult to physically realize, however, based on many practical limitations. There is no method to ensure that said device surrounding the torso would stay in place during activation or if it would move relative to said magnetic mats under the resistive force (stiffness) of the diaphragm and viscera themselves. If such a device were firmly clamped around the torso with the force necessary to hold it body-length-wise stationary, then expansion of the rib cage, which is a natural motion during breathing to allow air volume inspiration, would be entirely prohibited. Additionally, the force required from said electromagnetic device would be quite excessive, implying that a large electromagnetic coil would be needed, as well as levels of electrical current that would be fatal to any human or animal. Said electromagnet with such higher current would also become extremely hot, which poses problems when implemented in the preferred close proximity to the ribs. In order to avoid burning the torso, said electromagnet would likely need to be positioned far enough away from the torso that electromechanical inefficiency would be high. Indeed, the safety and electromechanical efficiency are competing design features.

An effective diaphragm assist device (DAD) should be able to impose a breathing pattern at a comfortable rate and volume, specific to a particular patient, in order to avoid feelings of shortness of breath or light-headedness, while allowing all normal respiratory dynamics to take place unimpeded. The end effect would be improved exercise tolerance and improved quality of life.

The present inventors have discovered that this is best-accomplished with a wholly or partially implantable DAD anchored to the skeletal system and employing a programmable actuator and kinematic mechanism mechanically-engaged directly to the diaphragm. This unique configuration may impart a periodic mechanical force profile having predetermined (programmable) cycles, in a similar manner as a conventional mechanical ventilator, or may be triggered by sensors to provide the necessary assistance to the diaphragm when the patient attempts to breathe. In certain diseases, the device may also feature an integrated cough assist function to facilitate airway clearance. It offers improved diaphragmatic assistance and, as a result, improved quality of life for patients suffering from any one of a host of diseases that affect respiration. The DAD will also be suitable for patients awaiting lung transplants and those who are being weaned from mechanical ventilation. Reduced health care costs and mortality rates are expected. Moreover, in terminal patients and those with diaphragmatic paralysis, such a device is able to completely control respiration.

### SUMMARY OF THE INVENTION

In accordance with the foregoing objects, the present invention is a diaphragm assist device (DAD) for assistance with inspiration and/or exhalation by physically-forced or aided diaphragm contraction and/or retraction, respectively, to assist patients to inspire and/or exhale. The device assists with respiration of patients having diaphragmatic dysfunction including those awaiting lung transplants, and those in need of weaning from mechanical ventilation.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a front perspective view of an exemplary DAD 2 according to the invention, in the preferred fully implantable embodiment above the diaphragm and on the right side.

FIG. 2 is a side view of the DAD 2 of FIG. 1, depicted in the abdomen, looking down to the diaphragm.

FIG. 3 is an enlarged top perspective view of the DAD 2 of FIGS. 1-2.

FIG. 4 is a block diagram of the DAD 2 of FIGS. 1-3.

FIG. 5 is a perspective front view of an exemplary DAD 12 according to an alternate embodiment of the invention, employing linear FAM actuators 20, again depicted in a cross section of the abdomen, looking up from below the diaphragm.

FIG. 6 is a block diagram of the DAD 12 of FIG. 5.

FIG. 7 shows a cross section view of an embodiment of a swaged fluidic artificial muscle actuator in a non-active state.

FIG. 8 shows a perspective view of the swaged fluidic artificial muscle actuator of FIG. 7.

FIG. 9 shows a cross section view of one embodiment of the end fittings of the swaged fluidic artificial muscle actuator.

FIG. 10 shows a cross section view of a second embodiment of the end fittings of the swaged fluidic artificial muscle actuator.

FIG. 11 is a graph illustrating a normal breathing pattern (top) which includes periods of inhalation, exhalation and automatic pause.

FIG. 12 shows an alternate embodiment in which the housing 4 is secured to the spine and the arms of linkage 6 and FAMs 20 are directed inward toward the paddles 10a, 10b.

FIG. 13 shows yet another alternate embodiment in which two separate DADs 2 are used, which are secured to the ribs.

FIG. 14 shows another alternate embodiment in which a single kidney-shaped paddle 10 covers both the right and left leaflets of the diaphragm and is actuated from the DAD system 2 mounted on the sternum.

FIG. 15 shows another alternate embodiment in which two oval paddles 10a, 10b cover the respective right and left leaflets of the diaphragm, and both are actuated from a DAD system 2 mounted on the spine.

FIG. 16 is a perspective front view of an exemplary DAD 120 according to an alternate embodiment of the invention, employing a balloon insufflation-type actuator 125, depicted in a cross section of the abdomen, looking down from above the diaphragm.

FIG. 17 is a block diagram of the DAD 120 of FIG. 16.

FIG. 18 is a graphical illustration of the improvement trend expected in the cost of breathing using the present device in select COPD patients.

### DETAILED DESCRIPTION OF THE PREFERRED EMBODIMENTS

The present invention is a diaphragm assist device (DAD) available as a fully or partially implantable unit, for physically promoting diaphragm contraction and/or retraction to assist patients to inspire and/or exhale.

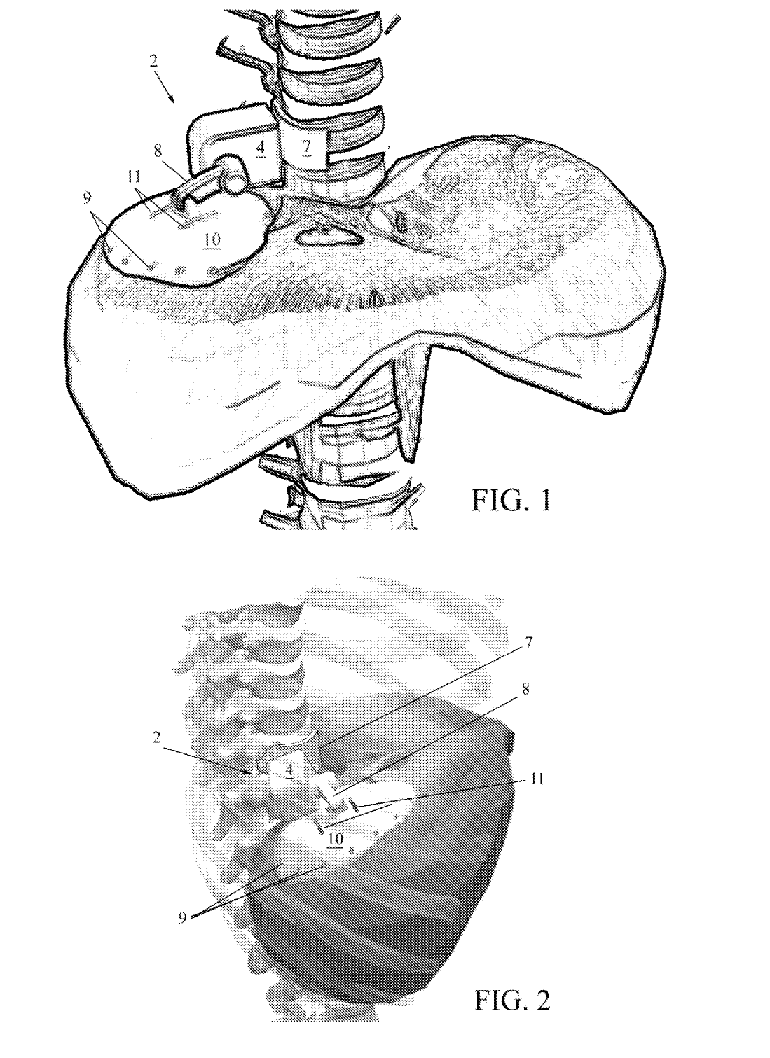

As seen in FIG. 1, a preferred embodiment of the DAD 2 comprises a housing 4 enclosing a programmable controller with memory and other supporting circuitry, a power source (e.g., battery), and an actuator, or “kinematic mechanism”, in communication with the programmable controller, and an articulating linkage 8 here including a single arm, the arm extending to paddle 10, which bears directly against and is affixed to the upper side of the diaphragm, installed on the right side, within the thoracic cavity.

Housing 4 is flat, smooth, as small as the electronics and actuator allow, and may be implanted (mounted to the skeletal system such as the spine) or mounted external. If mounted external the housing 4 is preferably separated into two distinct housings 4A, 4B, one 4A externally wearable or in close proximity to the body and containing the programmable controller, memory, supporting circuitry, and power source, and connected by wires via a port or conduit into the body to a second housing 4B containing the kinematic mechanism and preferably mounted to the skeletal system (e.g., spine, sternum or rib cage). The kinematic mechanism may likewise be external such that the articulating linkage enters the abdomen through a port, but is more preferably internal (e.g., implanted) as described above. In either case the anchor point of at least part of the kinematic mechanism is the skeletal system.

The kinematic mechanism may include an electro-mechanical actuator operatively coupled to articulating linkage 8, or alternatively hydraulic and/or pneumatic fluidic artificial muscles (to be described), or an electroactive polymer, piezo-electric material or shape memory-alloy such as Nitinol®.

FIG. 2 is a side view of the DAD 2 of FIG. 1 in the abdomen looking down. The DAD 2 is mounted to the spine above the diaphragm and on the right side within the thoracic cavity, with the ribs appearing semi-transparent. The spinal mounting is accomplished with an annular semi-circular half-collar 7 affixed to the housing 4 and directed inwardly toward the spine for embracing it.

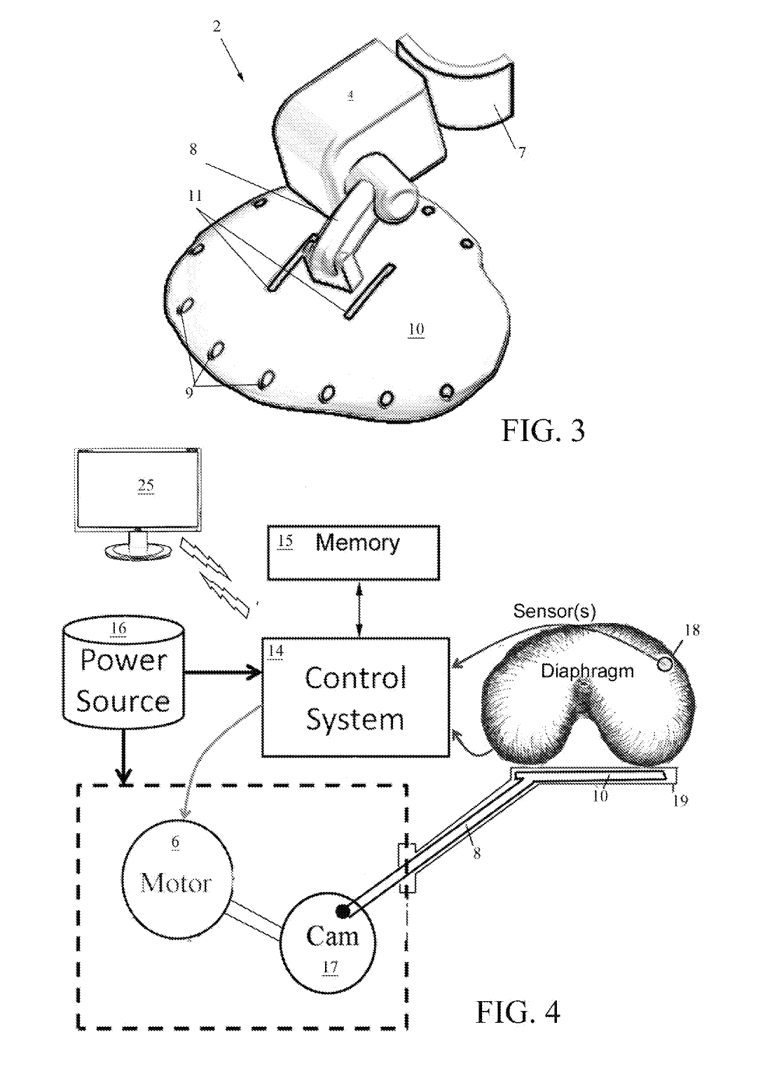

FIG. 3 is an enlarged perspective front view of the DAD 2 of FIGS. 1-2. As seen in this embodiment, the housing 4 has smooth, rounded edges and contains the majority of the parts of the DAD 2. The articulating linkage 8 has an anatomically conformal shape as it attaches to the kinematic mechanism within the housing 4 at one end and to the paddle 10 at the other end. The paddle 10, while generally in an oval-like shape, is shaped to mimic the actual anatomical dome shape of a healthy diaphragm.

FIG. 4 shows the programmable control system 14 with memory 15, power source (e.g., battery) 16 and supporting circuitry, and kinematic mechanism 6. The articulating linkage 8 is formed as an arm 8 (one being shown but two are possible) extending to paddle 10, the paddle 10 being mechanically-engaged directly to the diaphragm. The arm of linkage 8 may be elastically or mechanically hinged to the housing 4 and extend in an anatomically preferred manner to paddle 10. As shown in FIG. 4, the arm of linkage 8 together with attached paddle 10 may be encased in rubberized (e.g., silicon rubber) shell 19 which is grommetted to the housing 4 so as to ensure a fluid-tight sterile seal.

The paddle 10 is preferably stiff relative to body tissue such that articulation of the paddle 10 pushes on abdominal contents, similar to the natural diaphragm function, to increase abdominal pressure, thereby contributing to expansion of the rib cage, increasing intrathoracic volume, and thus facilitating inspiration. However, the stiffness of paddle 10 may allow it to flex a preferred amount in response to its downward pushing on the abdominal contents. Additionally, the paddle 10 is sufficiently soft and compliant so as not to traumatize the diaphragm (or other anatomic structures that the paddle might come into contact with). Each paddle 10 is affixed directly to the diaphragm, preferably physically attached to the upper side of the diaphragm (e.g., the central tendon of diaphragm) with surgical adhesive, or by mechanical means such as sutures, staples, or other known connecting means. Affixing the paddle 10 to the diaphragm may be achieved by stitching through the exemplary series of holes 9 along the perimeter of the component as illustrated. Additionally, part of the diaphragm may be taken up through the exemplary slots 11 to assist in restoring the natural shape of the diaphragm, should it become slack as a consequence of its disease process. Alternatively, the paddle 10 may utilize implantable silicon gel pads such as shell 9 which stick to the diaphragm, in which case the diaphragm will move with the paddle 10 via suction or adhesion.

The kinematic mechanism of FIG. 4 comprises an electro-mechanical actuator, and more specifically a brushless DC rotary step motor 6 connected to a cam mechanism 17, though any suitable implantable rotary actuator for producing a rotary motion or torque may be used. For example, induction motors, brushed DC motors, brushless DC motors, servo or stepper motors, or reluctance motors may be used.

The arm of linkage 8 is connected to one side of cam shaft 17 offset from its axis of rotation, and motor 6 is gear-coupled to cam shaft 17 to rotate it. Thus, controlled rotation of motor 6 rotates cam shaft 17 which in turn drives paddle 10, raising and lowering it in a fan-like motion. In this case cam shaft 17, any associated bearings, other mechanical components, linkage arm(s) 8, and paddle(s) 10 comprise the kinematic mechanism.

In addition, one or more sensors 18 may be connected to the programmable control system 14 to provide operational feedback. Sensors 18 are connected to the programmable control system 14 to provide operational feedback, though in another embodiment, feedback, and hence the sensors, may not be necessary. The sensor(s) 18 may include any one or more from among the following types of sensors:
respiratory function sensor, such as an optical fiber using Bragg grating (FBG) sensing for monitoring respiratory function.
a movement/position sensor to detect respiration or attempted respiration based on movement of the rib cage or diaphragm, whether that movement be displacement, velocity, or acceleration.
a movement/position sensor to detect movement of the diaphragm (dimensional changes may be measured by variations in resistance, inductance, capacitance, and piezoelectric effect).
A load sensor to detect muscle load of the diaphragm.
an air flow sensor to detect respiration or attempted respiration based on air flow within the respiratory system, including, but not limited to, the nasal cavity, mouth, trachea, and bronchioles.
a heart rate sensor to detect respiration or attempted respiration based on heart rate or changes thereof.
a temperature sensor to detect respiration or attempted respiration based on temperature change of a portion of the patient's body.
an electrical sensor to detect electrical potential of muscles or nerves, e.g., EMG and EEG.
a blood/oxygen sensor to detect blood oxygen levels.
a pressure sensor, such as a solid-state micromanometric pressure sensor for measuring intrathoracic pressure, or other pressure sensing in the upper/lower respiratory tract. This can be used for particularly advantageous sensing of negative pressures that signal the initiation of a breath.

All components of the fluid control system 12 and programmable control system 14 are powered by a conventional power supply 16, which may be a rechargeable battery and regulator circuit.

Power source 16 may be a conventional lithium ion or Li/CFx battery designed for medical applications. Optionally, power source 16 may include a kinetic charging system for converting a patient's kinetic energy from movement directly into electrical energy for recharging the battery. In the illustrated embodiment all the foregoing components of DAD 2 including programmable control system 14, memory 15 (see FIG. 4), power source (e.g., battery) 16, kinematic mechanism 6 and 15, articulating linkage 8 and paddle(s) 10 are configured in a unitary implantable package, though one skilled in the art will readily understand that programmable control system 14, and/or power supply 16 and/or kinematic mechanism components 6 and 15 may be remotely located outside the body. One skilled in the art should understand that the components described above may be separate or part of one unit/housing, located either inside or outside the body, or a combination thereof.

In addition, a remotely-located video display and control center 25 is in communication by bluetooth or other suitable wired or wireless communication protocol with the programmable control system 14 to provide a patient, provider, and/or caretaker interface for programming mechanical force profiles, control, and viewing of the imparted profile as well as feedback from sensor(s) 18 for sensing device 2 function (e.g., to ensure that the device is operating as it is supposed to) and/or patient physiology (e.g., the tidal volume generated or other physiological parameter(s)).

One skilled in the art will also understand that actuator 6 may alternatively comprise at least one linear electromechanical actuator, such as solenoids, for direct linear push/pull of paddle(s) 10.

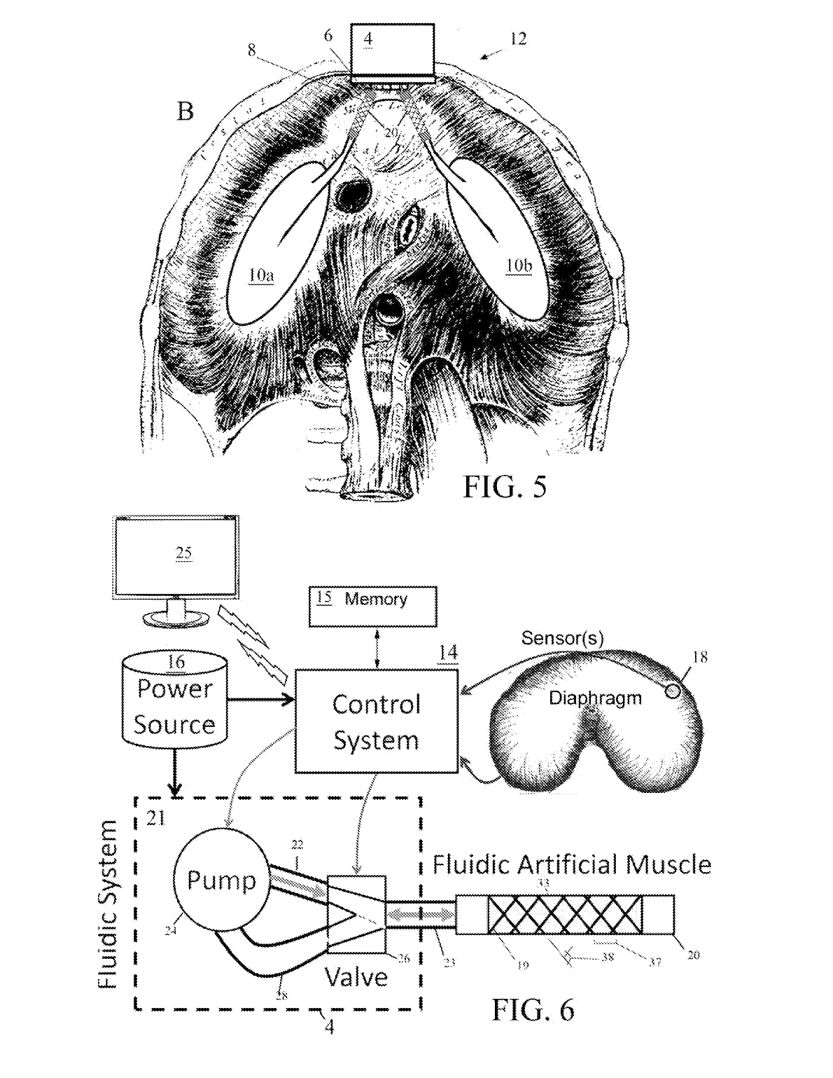

In an alternative embodiment of DAD 12 shown in FIGS. 5-6 the kinematic mechanism comprises a fluidic system 21 including a hydraulic pump 24 in fluid communication with two linear actuators, both fluidic artificial muscles 20, and both coupled between the housing 4 and one of the arms of linkage 8 for leveraged pivoting thereof. In the embodiment of FIG. 5 the housing 4 is mounted on the sternum, and the paddles are extended therefrom to the diaphragm.

In both embodiments of FIGS. 1-4 and 5-6 the kinematic mechanism is operable on the diaphragm through the linkage 8 and paddle(s) 10a, 10b to impart a predetermined mechanical load profile comprising programmable dynamic loading cycles stored in the memory 15 of controller 14. This selectively assists during inspiration and/or expiration of patients. While the embodiment of FIGS. 1-4 shows the DAD 12 on the upper surface of the diaphragm, this is not intended to be a limitation of the invention as it may be equivalently be placed on the lower surface of the diaphragm as in FIGS. 5 and 12-15. These illustrations are meant merely to teach the breadth of possible implantation options of the invention, which may be tailored to each individual patient.

FIG. 6 is a schematic drawing of the embodiment of FIG. 5, in which the linear actuators 20 comprise fluidic artificial muscles (FAMs) in fluid communication 23 with a fluid control system 21. Although in FIG. 6 one fluidic artificial muscle (FAM) 20 is shown, multiple FAMs 20 may preferably be used. Each FAM 20 is coupled from housing 4 to an arm of the articulating linkage 8 (not pictured). The FAM actuators 20 are in fluid communication via conduit 23 with a fluid control system 21, the latter including a pump 24 connected by inflow conduit 28 and outflow conduit 22 through an electrically-controlled valve 26. Both pump 24 and valve 26 are in electrical communication with programmable control system 14 for selectively pumping fluid into and out of FAMs 20 in a controlled manner.

One of the distinguishing features of the above-described system is that a biocompatible fluid is used inside FAMs 20 rather than air or oil. Any suitable fluid is considered within the scope and spirit of the invention. However, a biocompatible fluid makes FAM 20 safer and better-suited for implantation. The biocompatible fluid may be, for example, a saline solution. This way, if a leak develops, air is not released inside the body, nor is any other harmful liquid or gas.

Again, one or more sensors 18 (described above) may be connected to the programmable control system 14 to provide operational feedback.

All the foregoing components of DAD 2 including FAM 20, fluid control system 21, programmable control system 14, and power supply 16 are configured in a unitary implantable package, though one skilled in the art will readily understand that only FAM 20 and sensors 18 are necessarily implanted and any one or more of the fluid control system 21, programmable control system 14, and/or power supply 16 may be remotely located outside the body. Again, the various components described may be separate or part of one unit/housing, located either inside or outside the body, or a combination thereof. The FAM 20 is in direct mechanical contact via linkage 8 and paddles 10a, 10b with the patient's diaphragm muscle imparting a selected mechanical force profile stored in memory 15, and mechanical stimulation in accordance with the selected profile (described below). Direct coupling to the diaphragm muscle is believed to help prevent barotrauma, volutrauma, and other complications caused by positive pressure devices. Again, remotely-located video display and control center 25 is in communication by bluetooth or other suitable wired or wireless communication protocol with the programmable control system 14 to provide a patient, provider, and/or caretaker interface.

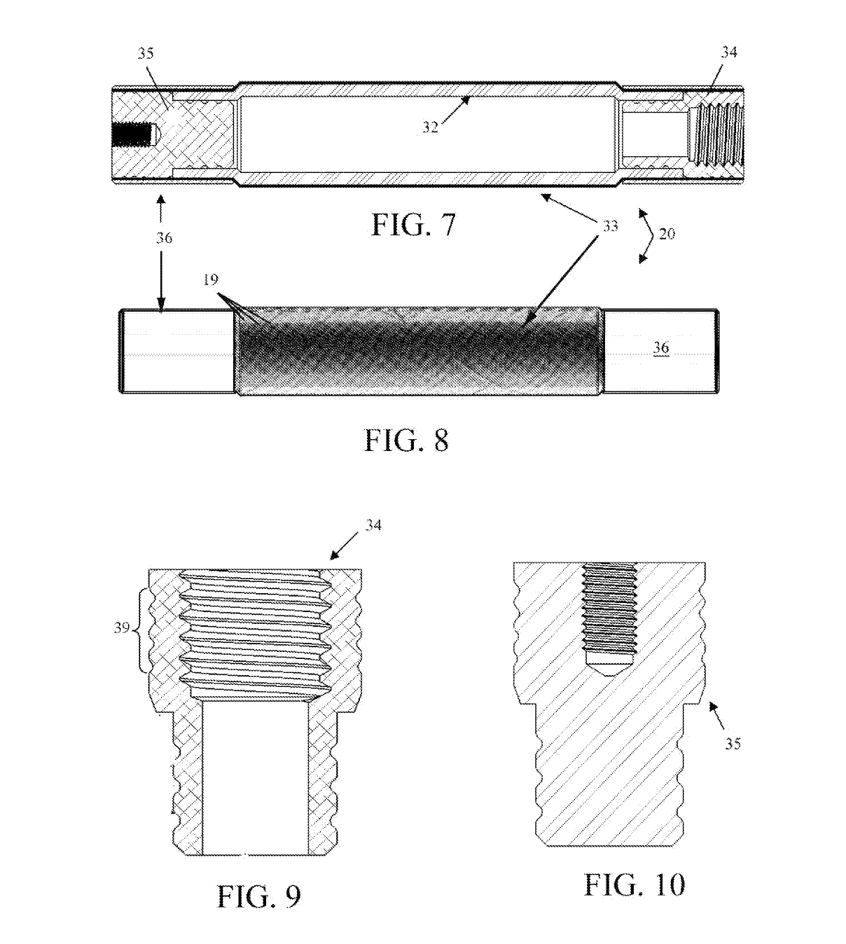

FIGS. 7-8 are more detailed illustrations of the FAM 20 used in the DAD 12 of FIGS. 5-6. The fluidic artificial muscle (FAM) actuator 20 (also known as an artificial muscle actuator, or McKibben artificial muscle, among other names) is a mechanical actuator that harnesses pressurized fluid to generate axial deflections. The FAM 20 generally comprises an inner elastic fluid bladder 32 surrounded by a relatively stiff braided sleeve 33. End fittings 34, 35 are attached to each end to seal the bladder 32 and allow for connection of the FAM actuator 20 to the fluid control system 21. In the illustrated embodiment a swage tube 36 is fitted around both end fittings 34, 35 sandwiching the braided sleeve 33 and bladder 32 as shown. The swage tubes 36 have a constant wall-thickness and constant-diameter, and upon fitting are plastically deformed to provide a fluid seal and a strong mechanical connection. The end fittings 34, 35 are primarily cylindrical and feature a stepped outer diameter, with two or more different diameters along their length. The stepped diameters create separate clamping regions for the braided sleeve 33 and bladder 32 that allows for different braid/bladder thicknesses and different amounts of compression.

The braided sleeve 33 in the above-described embodiment preferably comprises fiber filaments 19 braided in a helical fashion to form a sleeve that can expand or contract in diameter. The sleeve 33 may alternatively be comprised of different layers of helically wrapped filaments that are stacked instead of woven, for example, in the case of two layers the two individual layers encircle the bladder in opposing directions. The filaments may be embedded into a soft (e.g. elastomer or rubber) matrix to maintain the spacing between fibers. Suitable filament materials include but are not limited to aramid, para-aramid, carbon, or fiberglass fibers.

Viewing FIG. 8, the sleeve filament density (distance between filaments 19) and initial braid angle of the filaments 19 of sleeve 33 can be varied to influence the stiffness, force generation, deflection range, and other important actuator properties. The initial braid angle of the filaments 19 is defined as the angle between a braid filament 19 and the radial axis of the actuator 20 when the sleeve 33 is tight against the pressure bladder 32 and the FAM actuator 20 is at its resting length (no internal pressure, no external loading).

As seen in FIG. 7, the pressure bladder 32 will preferably be made from a low modulus, elastic material such as an elastomer or rubber. Silicone, polyurethane, and latex rubbers are the preferred materials, although any suitable material may be used without changing the invention. These materials allow for the large strains associated with pressurization, while minimizing the amount of energy required in order to pressurize them. Wall thickness of the bladder 32 is chosen to ensure that the operating pressure can safely be maintained without rupture.

The braided sleeve 33 and pressure bladder 32 may be made as a single combined component. This can be accomplished by co-curing of the elastic bladder 32 material and the filaments of the braided sleeve 33. All components of the FAM actuator 20 should be fabricated with biocompatible materials for the embodiment that requires surgical implantation. In an embodiment wherein the FAM actuators are not implanted, then biocompatible materials may not be necessary.

With combined reference to FIGS. 6-8, in operation the electrically-controlled valve 26 and fluid pump 24 serve to pressurize and depressurize the FAM actuator 20 under control of the control system 14 (see FIG. 6). The control system 14 uses information received by the sensor(s) 18 to detect physiological attributes such as patient respiratory function and to calculate how the patient's diaphragm may best be assisted. Pressurization will produce force and motion, either contractile or extensile, due to the interaction between the bladder 32 and braided sleeve 33 (FIGS. 7-8). The inner elastic bladder 32 is pressurized with a fluid such as air or saline solution, causing an inflation and expansion of the bladder 32. The braided sleeve 33 around the bladder 32 is thereby forced to expand. However, the fixed length of the stiff sleeve fibers generates a contractile or extensile force along the main axis of the actuator, in addition to relative motion between the two end fittings 34, 35. The direction of force and motion are dependent on the initial angle between the filaments 19 of the braided sleeve 33.

One skilled in the art should understand that FAMs 20 may alternatively be replaced by cylinder actuators, or wire lengths of an electroactive polymer, piezo-electric material or shape memory-alloy such as Nitinol®. Contraction and relaxation of Nitinol® depends on the temperature of the nitinol alloy wire. Nitinol© wire has a high electrical resistance approximately 1.25 ohms resistance per inch for a 6 mm wire, and so application of electrical current effectively makes it a linear actuator.

In the embodiment of FIGS. 1-4 electro-mechanical actuation is controlled by the controller 14, and in the embodiment of FIGS. 5-6 pressurization is similarly controlled by the controller 14. In both cases controller 14 imposes a mechanical force profile stored in the memory 15 of controller 14 which comprises a sequence of predetermined dynamic loading cycles. If desired, the controller 14 may be somewhat reactionary to sensors 18, wherein designed and programmed control algorithms adjust the operation of the device based on at least one sensor input. Accommodation for normal respiratory aberrations, such as coughing, sneezing, and holding one's breath, are also included in the function of the controller 14.

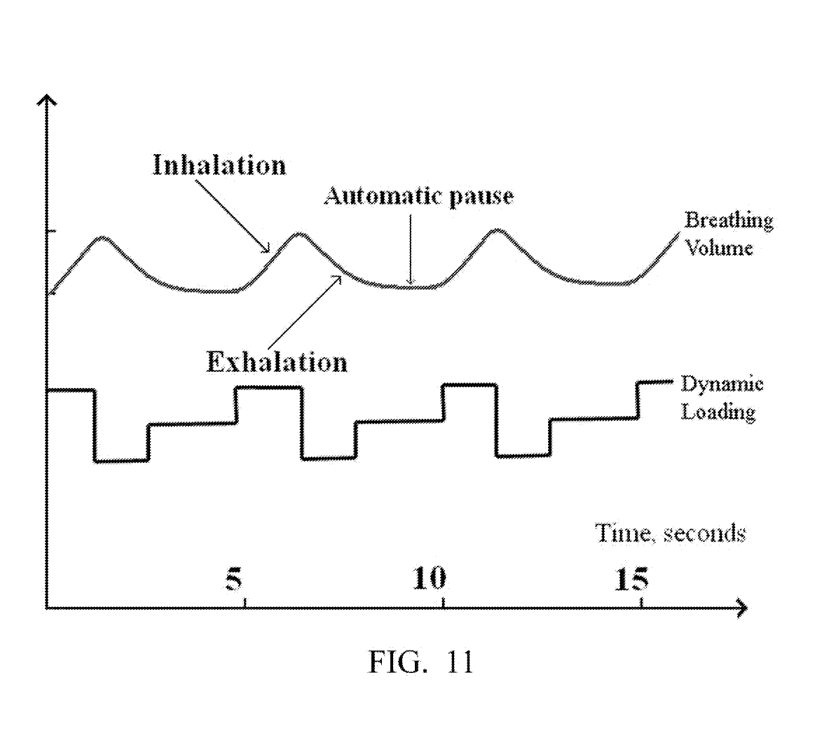

FIG. 11 is a graph illustrating a normal breathing pattern (top) which includes periods of inhalation, exhalation and automatic pause. To achieve this, the mechanical force profile (bottom) comprises a pulse wave or pulse train having a short positive duty cycle immediately followed by a short negative duty cycle, separated by a pause. This is purely an exemplary waveform, however, and is not meant to limit any of the aforementioned control directives or scenarios.

Another alternative (optional) mechanical force profile provides a cough assist function for patients with certain diseases that adversely affect airway clearance. The cough assist force profile may comprise an intermittent waveform having a more pronounced positive duty cycle to support a deep inspiration, followed by an abrupt negative duty cycle for forced expiration, separated by a shorter pause to replicate the abrupt expiratory flow and force of the patient's cough.

Still another mechanical force profile provides a training feature to strengthen or condition the diaphragm, prevent atrophy, and otherwise avoid ventilator-induced diaphragmatic dysfunction. A training profile may provide an incremental proportional assist to the diaphragm whereby the DAD begins by providing a relatively large assist and this assistance is reduced over time as the diaphragm gains strength. Additionally, a training profile may comprise much more prolonged duty cycles to train the patient to breathe deeply, rather than taking shallow chest breaths. Moreover, a training profile may comprise a negative resistance profile that resists movement of the diaphragm, temporarily increasing breathing difficulty and therefore strength.

As shown in FIGS. 1-6 the housing 4 is secured to some feature of the skeletal system as a bearing surface, preferably the spine, with the arm(s) of linkage 8 and FAM(s) 20 directed anatomically toward the paddle(s) 10 in a manner that preferably follows the contour of the diaphragm. The paddle(s) are preferably oval-like to conform to the domes of the right and left leaflets of the diaphragm. However, one skilled in the art should understand that the illustrated embodiments are meant merely to detail how and where the DAD 2 can be implanted, showing the approximate size of the device relative to the patient's body. The housing 4 may be implanted and secured to any suitable feature of the skeletal system including ribs, spine, sternum, pelvis, etc., and the components can be located above and/or below the diaphragm, with mechanical attachments thereto being on the upper and/or lower diaphragm surface, respectively.

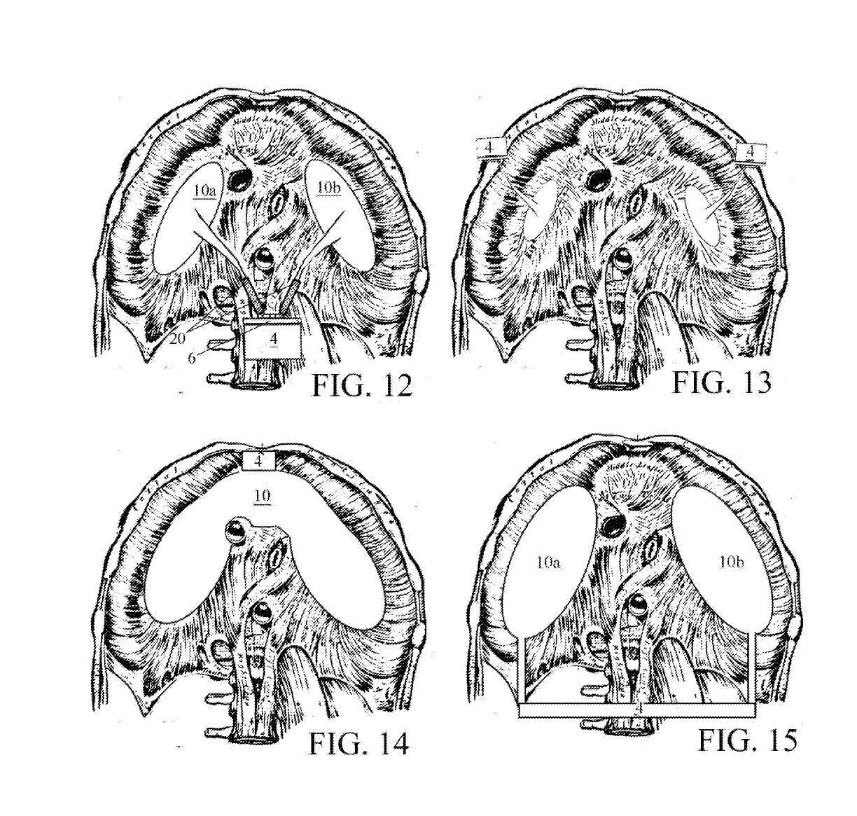

FIG. 12 shows an embodiment in which the housing 4 is secured to the spine and the arms of linkage 8 and FAMs 20 are directed toward the paddles 10a, 10b from below the diaphragm.

FIG. 13 shows an alternate embodiment in which two separate single-arm DADs 2 are used, each comprising an implantable housing 4 attached to one side of the ribs and wielding a single-arm linkage 8 and FAMs 20 directed toward a single paddle 10. The two separate DADs 2 each assist either the right or left leaflets of the diaphragm.

The paddle 10 or paddles 10a, 10b need not be oval or other suitable shapes, and a single paddle 10 or pair of paddles may be used.

FIG. 14 shows another alternate embodiment in which a single kidney-shaped paddle 10 covers both the right and left leaflets of the diaphragm, and is actuated from the housing 4 mounted on the sternum.

FIG. 15 shows yet another alternate embodiment in which two oval paddles 10a, 10b cover the respective right and left leaflets of the diaphragm, and both are actuated from an elongate housing 4 mounted on the spine.

In another embodiment the paddle 10 or paddles 10a, 10b may be eliminated, provided the linkage 8 can be attached directly to the right and/or left leaflets of the diaphragm, preferably with the direct attachment occurring through the central tendon of the diaphragm.

FIG. 16 is a perspective front view of an exemplary DAD 120 according to an alternate embodiment of the invention, employing a balloon insufflation-type actuator 125, depicted in a cross section of the abdomen, looking down from above the diaphragm. Similar to the embodiment of FIG. 5 the DAD 120 comprises a housing 4 enclosing a programmable controller with memory and other supporting circuitry, a power source (e.g., battery), and a kinematic mechanism in communication with the programmable controller. However, in this case the kinematic mechanism comprises a fluid control system including a pump inside housing 4 mounted on the spine, and in fluid communication via conduit 123 with an expandable member 125 in direct contact with the diaphragm. The fluid control system is operable on the diaphragm through the conduit 123 to inflate and/or deflate expandable member 125 to impart a predetermined mechanical load profile as described above. This selectively assists during inspiration and/or expiration of patients. While the embodiment of FIG. 16 shows the expandable member 125 on the upper surface of the diaphragm, this is not intended to be a limitation of the invention as it may be equivalently be placed on the lower surface of the diaphragm similar to FIGS. 5 and 12-15. The expandable member 125 comprises a balloon having an overall boomerang shape and, when inclated, an oval cross-section. Where necessary to provide a supporting surface, a fixed-position shroud 127 may be affixed to the housing 4 above (or in alternate embodiments below) expandable member 125 to serve as a bearing surface as expandable member 125 expands. The conduit 123 is coupled to the expandable member 125 by a coupling 127.

The expandable member 125 may be any implantable compliant material. Examples of compliant materials suitable for use in an expandable member 125 as described herein include, but are not limited to: silicone, mylar, latex rubber, polyurethane, nylon, polyethylene, polyester, polyamide and polyurethane.

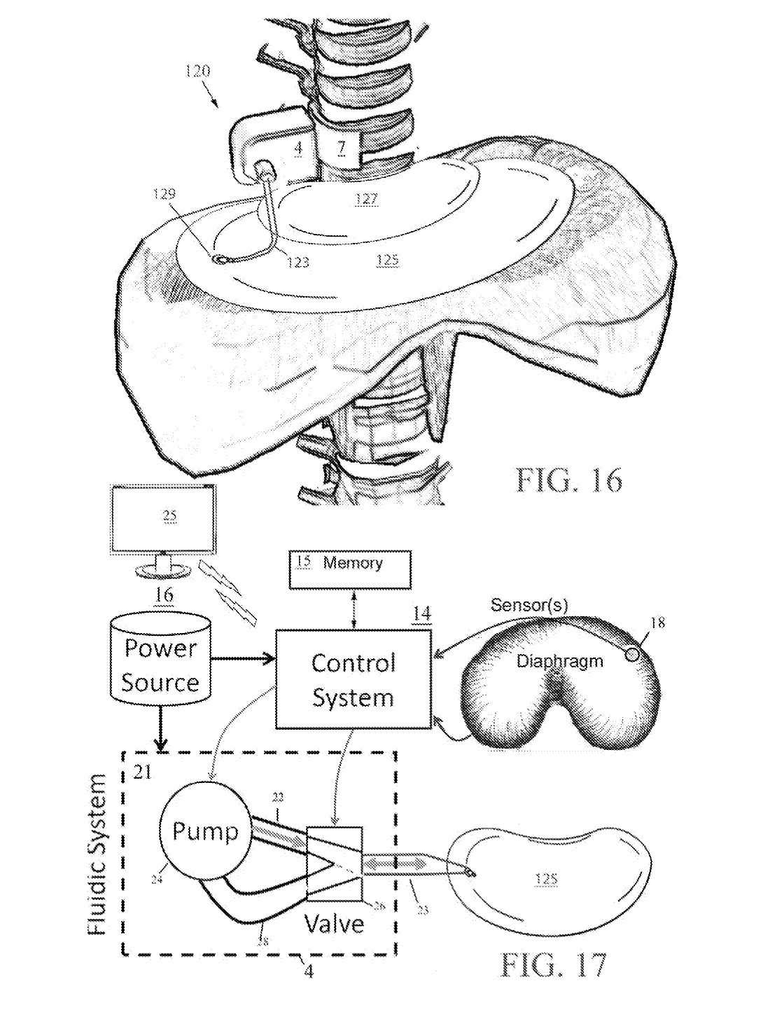

FIG. 17 is a schematic drawing of the embodiment of FIG. 16, in which the actuator comprises expandable member 125. The fluid control system including pump 24 inside housing 4 is substantially as described above with regard to FIG. 6. Both pump 24 and valve 26 are in electrical communication with programmable control system 14 for selectively pumping fluid into and out of expandable member 125 in a controlled manner.

In light of all the foregoing it should now be apparent that the above-described diaphragm assist device (DAD) will provide mechanical assistance to diaphragmatic function and facilitate respiration, with an optional cough assist function for patients with certain diseases that adversely affect airway clearance. This will be of great benefit to patients having diaphragmatic dysfunction including those awaiting lung transplants, and those in need of weaning from mechanical ventilation, with reduced oxygen cost of breathing in select patients.

If desirable, any of the above-described DAD devices may be used to supplement an existing high frequency oscillatory ventilator. There are different types of high frequency ventilation, and it is well-known that high frequency ventilation may be used alone, or in combination with conventional mechanical ventilation. The above-described DAD devices would take the place of, or work in concert with, conventional mechanical ventilation.

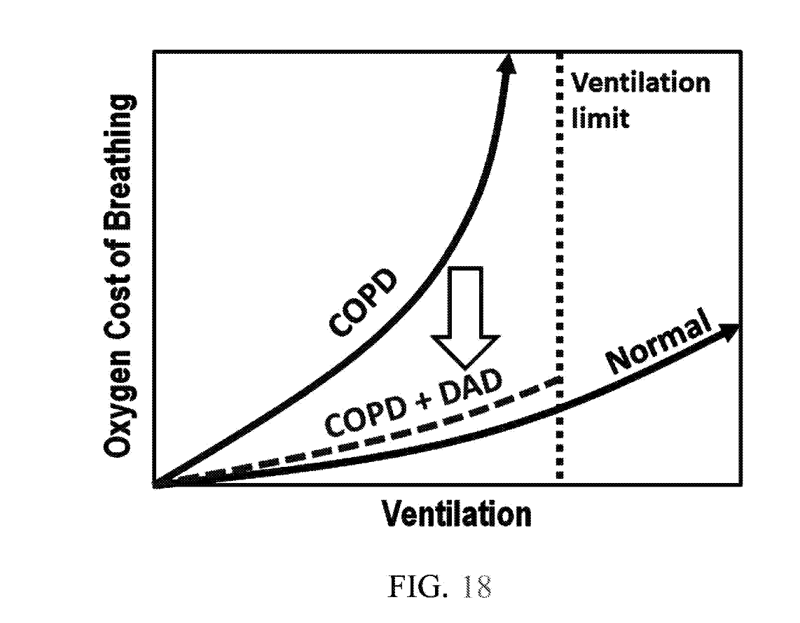

FIG. 18 is a graphical illustration of the improvement expected in the cost of breathing using the present device in select COPD patients. It is an illustrative graph of respiratory muscle oxygen consumption for a normal person and someone with COPD, as adapted from The Normal Lung by John F. Murray (2^{nd} edition, W.B. Saunders Company, Philadelphia, Pa., 1986, page 135), and modified with a curve representing the effect of the DAD device, as indicated by the arrow. This shows an improvement in the cost of breathing using such a device.

It should be noted, however, that the added curve is not based on known data points and is only intended to illustrate the effective concept of the invention, which is a shift from a disadvantaged diaphragm toward a normal diaphragm.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

### INDUSTRIAL APPLICABILITY

An effective diaphragm assist device (DAD) will impose and/or provide mechanical assistance to diaphragmatic function and facilitate respiration, with optional cough assist function for patients with certain diseases that adversely affect airway clearance. This is of great benefit to patients having diaphragmatic dysfunction including those awaiting lung transplants, and those in need of weaning from mechanical ventilation. The DAD reduces the oxygen cost of breathing, and may improve exercise tolerance and quality of life.

What is claimed is:

1. A diaphragm-assist device for assisting in inspiration and/or exhalation of a patient, comprising:
a control system, said control system outputting instructions through a command signal,
a power source,
an actuator in communication with said controller for converting said command signal into a mechanical load profile;
a kinematic linkage mechanically coupled between said actuator and the diaphragm of said patient for directly mechanically loading said diaphragm muscle in accordance with said mechanical load profile.

2. The diaphragm-assist device according to claim 1, wherein said mechanical load profile comprises a wave function.

3. The diaphragm-assist device according to claim 2, wherein said mechanical load profile comprises a cough assist profile.

4. The diaphragm-assist device according to claim 2, wherein said mechanical load profile comprises a training profile to condition the diaphragm.

5. The diaphragm-assist device according to claim 1, further comprising a remote video display and control center in communication with said control system for providing a user interface.

6. The diaphragm-assist device according to claim 1, wherein said power source comprises a battery and kinetic charging circuit for recharging the battery.

7. The diaphragm-assist device according to claim 1, wherein said kinematic linkage comprises at least one articulating arm extending to said diaphragm.

8. The diaphragm-assist device according to claim 6, wherein said at least one arm comprises a pair of arms extending to opposing leaflets of said diaphragm.

9. The diaphragm-assist device according to claim 1, further comprising at least one paddle attached to said diaphragm.

10. The diaphragm-assist device according to claim 8, wherein said at least one paddle comprises a pair of paddles attached to opposing leaflets of said diaphragm.

11. The diaphragm-assist device according to claim 8, wherein said kinematic linkage comprises at least one arm extending to said at least one paddle.

12. The diaphragm-assist device according to claim 1, further comprising at least one sensor in communication with said programmable controller for sensing an operational performance metric.

13. The diaphragm-assist device according to claim 1, wherein said actuator comprises a linear actuator.

14. The diaphragm-assist device according to claim 12, wherein said linear actuator comprises a fluidic artificial muscle.

15. The diaphragm-assist device according to claim 1, wherein said actuator comprises a rotary actuator.

16. The diaphragm-assist device according to claim 14, wherein said rotary actuator comprises a motor.

17. The diaphragm assist device of claim 13, further comprising a fluidic system including a pump and valve for selectively enabling pressurization and depressurization of said fluidic artificial muscle.

18. The diaphragm assist device of claim 17, wherein said fluidic system comprises a biocompatible fluid.

19. The diaphragm assist device of claim 12, wherein said at least one sensor detects any one from among the group consisting of respiratory function, movement of the rib cage, intra-abdominal (below diaphragm) pressure, movement of the diaphragm, muscle load of the diaphragm, intra-thoracic (above diaphragm) pressure, air flow and pressure within the respiratory system, cardiac cycle, temperature, electrical potential of a muscle or nerve, and blood oxygen level.

20. A diaphragm-assist device for assisting in respiration of a patient, comprising:
a control system, said control system including,
non-transitory computer memory, and a programmable controller programmed with control software comprising computer instructions stored on said non-transitory computer memory for outputting an electrical signal,
a power source,
an actuator in communication with said controller for converting said command signal into a mechanical load profile;
at least one paddle in physical proximity to said patient's diaphragm; and
a kinematic linkage mechanically coupled between said actuator and said at least one paddle.

21. The diaphragm-assist device according to claim 20, wherein said mechanical load profile comprises a wave function.

22. The diaphragm-assist device according to claim 21, wherein said mechanical load profile comprises a cough-assist profile.

23. The diaphragm-assist device according to claim 21, wherein said mechanical load profile comprises a training profile to condition the diaphragm.

24. The diaphragm-assist device according to claim 20, further comprising a remote video display and control center in communication with said control system for providing a user interface.

25. The diaphragm-assist device according to claim 20, wherein said power source comprises a battery and kinetic charging circuit for recharging the battery.

26. The diaphragm-assist device according to claim 20, wherein said kinematic linkage comprises at least one arm extending to said diaphragm.

27. The diaphragm-assist device according to claim 20, wherein said at least one paddle comprises a pair of paddles attached to opposing leaflets of said diaphragm, and said kinematic linkage comprises two arms, each attached to one of said pair of paddles.

28. The diaphragm-assist device according to claim 20, further comprising at least one sensor in communication with said programmable controller for sensing an operational performance metric.

29. The diaphragm-assist device according to claim 20, wherein said actuator comprises a linear actuator.

30. The diaphragm-assist device according to claim 20, wherein said actuator comprises a rotary actuator.

31. A method of improving respiratory function in a patient using a diaphragm assist device comprising a control system for outputting a command signal, a power source, an actuator in communication with said controller for converting said command signal into a mechanical load profile, and a kinematic linkage connected to said actuator for imparting a mechanical load in accordance with said mechanical load profile, said method comprising the steps of:
implanting at least one part of said diaphragm assist device inside a patient's body; and
operating said diaphragm assist device to produce a command signal defining a mechanical load profile comprising a wave function; and
delivering said mechanical loading directly to said patient's diaphragm in accordance with said mechanical load profile via the implanted part of said diaphragm assist device, thereby allowing reducing the oxygen cost of breathing for select patients.

32. A device for assisting respiration of a patient, comprising:
a control system that commands a respiratory function;
an actuator in communication with said control system for converting said command signal into a mechanical load;
a power source;
an implanted linkage mechanically coupled between said actuator and the patient's diaphragm.

33. The respiratory assist device of claim 32, further comprising a kinematic mechanism operably connected between said actuator and said implanted linkage.

34. The respiratory assist device of claim 32, further comprising at least one sensor to detect respiration or attempted respiration.

35. A device for regulating respiration of a patient, comprising:
a control system that commands a respiratory function;
an actuator in communication with said control system for converting said command signal into a mechanical load;
a power source;
an implanted linkage mechanically coupled between said actuator and the patient's diaphragm.

36. The respiratory regulating device of claim 35, further comprising a kinematic mechanism operably connected between said actuator and said implanted linkage.

37. A device for assisting respiration of a patient, comprising:
a control system that commands a respiratory function;
a power source;
a wholly implanted mechanical actuator, in communication with said control system for converting said command signal into a mechanical load.

38. The diaphragm assist device of claim 37, further comprising a kinematic mechanism for translating said mechanical load directly to said patient's diaphragm.
